(19) United States
(12) Plant Patent Application Publication
(10) Pub. No.: US 2016/0286706 P1
(43) Pub. Date: Sep. 29, 2016

(54) Nepenthes 'CANTNEPSUK'

(50) ;

(71) Applicant: Robert Cantley, Samagi Mawatha (LK)

(72) Inventor: Robert Cantley, Samagi Mawatha (LK)

(21) Appl. No.: 14/998,640

(22) Filed: Jan. 22, 2016

(30) Foreign Application Priority Data
Mar. 23, 2015 (QZ) 2015/0644

(51) Int. Cl. A01H 5/00

(52) U.S. Cl. PLT/373

(57) ABSTRACT

A new and distinct Nepenthes Rafflesiana×sibuyansis cultivar named ‘CANTNEPSUK’ is disclosed, characterized by exceptionally rapid growth. Plants tolerate a broad range of temperature and humidity, and can tolerate roots fully submerged in water. Ornamental pitchers are squat and uniquely dark purple and yellow. Plants form long tendrils. The new variety is a Nepenthes rafflesiana×sibuyansis, normally produced as an ornamental indoor plant.

### LATIN NAME OF THE GENUS AND SPECIES

Nepenthes rafflesiana×sibuyansis

### VARIETY DENOMINATION

‘CANTNEPSUK’

### BACKGROUND OF THE INVENTION

The new Nepenthes rafflesiana×sibuyansis cultivar is the product of a planned breeding program conducted by the inventor, Robert Cantley, in Lindula, Sri Lanka. The objective of the breeding program was to produce novel Nepenthes varieties for ornamental commercial applications. The cross resulting in this new variety was made December of 2008.

The seed parent is the unpatented unnamed Nepenthes rafflesiana. The pollen parent is unpatented unnamed Nepenthes sibuyansis. The new variety was selected May 18, 2011 by the inventor at a commercial nursery in Lindula, Sri Lanka.

Asexual reproduction of the selected new cultivar was performed by meristem tissue culture after selection in 2011. Asexual reproduction of this variety was first performed at a commercial laboratory in Lindula, Sri Lanka. Subsequently, many generations have been produced via meristem tissue culture.

### SUMMARY OF THE INVENTION

The cultivar ‘CANTNEPSUK’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘CANTNEPSUK’. These characteristics in combination distinguish ‘CANTNEPSUK’ as a new and distinct Rafflesiana×sibuyansis cultivar:

1. Exceptionally rapid growth.
2. Broad range of temperature tolerance
3. Increased tolerance of lower humidity for pitcher production.
4. Plants can be grown with roots completely submerged in water, allowing for aquaponics and a other alternative media.
5. Mature plants form pitchers colored near Purple N77A and Greyed-Purple 187A.
6. Squat picture shape.
7. Pitcher wing coloration including Purple N77A, Greyed-Purple 187A and Yellow-Green 145C.
8. Distinctive rib formation on peristome.
9. Pitcher lid coloration near Yellow 2D.
10. Long tendrils.

Plants of the new cultivar ‘CANTNEPSUK’ are similar to plants of the seed parent, Nepenthes rafflesiana in some horticultural characteristics. Plants of the new cultivar ‘CANTNEPSUK’ however, have a significantly different combination of phenotypic traits. Some of these differences include:

1. The new variety reliably produces pitchers in lower humidity situations.
2. The new variety has more purple coloration throughout pitcher.
3. The new variety tolerates a wider temperature range than the seed parent.
4. The new variety has a shorter pitcher length.

Plants of the new cultivar ‘CANTNEPSUK’ are similar to plants of the pollen parent, Nepenthes sibuyansis in some horticultural characteristics. Plants of the new cultivar ‘CANTNEPSUK’ however, have a significantly different combination of phenotypic traits. Some of these differences include:

1. The new variety reliably produces pitchers in lower humidity situations.
2. The new variety has a purple pitcher lid, compared to typical red pitcher lid of the pollen parent species.
3. The new variety has longer tendrils.
4. The new variety tolerates a wider temperature range than the seed parent.

### COMMERCIAL COMPARISON

Plants of the new cultivar ‘CANTNEPSUK’ are comparable to the unpatented commercial variety Nepenthes ‘Linda’. The two varieties are similar in some horticultural characteristics; however, the new variety ‘CANTNEPSUK’ differs in the following:

1. Pitcher color of the new variety is purple to greyed-purple whereas pitcher color of this comparator is red to greyed-red.
2. The new variety reliably produces pitchers in lower humidity situations.
3. The new variety tolerates a wider temperature range than the seed parent.
4. The new variety has a shorter pitcher length.
5. The new variety has longer tendrils.

Plants of the new cultivar ‘CANTNEPSUK’ can also be compared to the unpatented commercial variety Nepenthes rafflesiana×sibuyansis ‘Habana’ (U.S. Plant Pat. No. 2,4643 P3). The two Nepenthes varieties are similar in most horticultural characteristics; however, the new variety ‘CANTNEPSUK’ differs in the following:

1. Pitcher color of the new variety is purple to greyed-purple whereas pitcher color of this comparator is red to greyed-red.
2. The new variety reliably produces pitchers in lower humidity situations.
3. The new variety tolerates a wider temperature range than the seed parent.
4. The new variety has a shorter pitcher length.
5. The new variety has longer tendrils.

### BRIEF DESCRIPTION OF THE PHOTOGRAPH

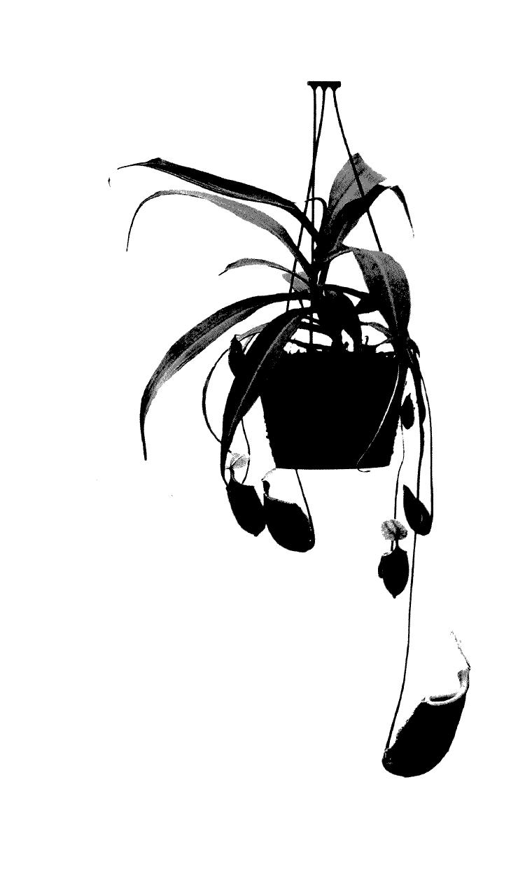

The accompanying photograph in FIG. 1 illustrates in full color a typical plant of ‘CANTNEPSUK’ grown in a greenhouse, in a 13 cm basket, with urns. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘CANTNEPSUK’ plants grown in a shade and insect screened nursery. The growing temperature ranged from 25° C. to 30° C. day and rom 12° C. to 15° C. during the night. Plants were shaded with 70% shadecloth. The nursery elevationis 4,500 feet (1450 m) and 5 deg north of the equator. No supplemental light given. Measurements and numerical values represent averages of typical plant types.
Botanical classification: Nepenthes rafflesiana×sibuyansis ‘CANTNEPSUK’
Propagation:
   Type of propagation typically used.—Meristem tissue culture.
   Time to initiate roots.—About 42 days at approximately 25° C.
   Time to produce rooted plantlet.—Approximately 16 weeks at approximately 20° C. to 30° C.
Plant:
   Age of plant described.—Approximately 3 years, mature specimen.
   Container size of the plant described.—8 inch.
   Overall plant shape or growth habit.—Single stem, outwardly arching leaves. Radially occurring pitchers hanging from plant.
   Plant height.—Average 60 cm.
   Plant diameter.—Average 64 cm.
   Growth rate.—Plants approximately 7 cm tall can grow approximately 1 cm per month in diameter. This growth continues up to 20 cm approximately. This can significantly vary with the use of fertilizers and different growing media.
   Branching characteristics.—No branching. Leaves emerge in immature plants directly from the base of the plant. The growing tip vines upwards and develops into a new leaf and a vining stem develops with inter-nodal distance between each new leaf.
   Number of leaves per plant.—Approximately 12 leaves in 3 yr. old specimen with basal shoots.
Foliage:
   Leaf.—Arrangement: Whorled. Quantity: Approximately 10 per main branch. Average Length: Mature length approximately 38.5 cm. Average Width: Approximately 5.0 cm. Shape of blade: Narrow elliptic. Apex: Acuminate, developing into tendril. Base: Attenuate. Attachment: Petiolate. Margin: Entire. Texture of top surface: Smooth, 4 longitudinal veins, occurring 2 on each side of the mid rib. Texture of bottom surface: Smooth with sparse, irregularly occurring nectar glands. Plant Leaf Color: Young foliage upper side: Near RHS Young foliage under side: Near RHS Mature foliage upper side: Near RHS Mature foliage under side: Near RHS Venation: Type: Parallel Venation color upper side: Near RHS Venation color under side: Near RHS Petiole: Average Length: Approximately 1.5-2 cm. Basal leaf petiole around 5 cm. Diameter: Approximately 0.15 cm. Color: Near RHS Yellow-Green 146D.
Flower: True flowers not observed. Flowering occurs only in older plants. Pitcher/Urn
   Longevity on plant.—
   Quantity of pitchers.—3 to 4.
   Shape.—Asymmetrical, squat urn.
   Urn.—Height: Approximately 16 cm. Width: Approximately 6 cm.
Reproductive organs: Not observed to date.
Other characteristics: Tolerance for a wider range of temperatures than typical of Nepenthes. Tolerance of roots in sitting water. Tolerance for lower humidity without a stop of pitcher production.

What is claimed is:

1. A new and distinct cultivar of Rafflesiana×sibuyansis plant named ‘CANTNEPSUK’ as herein illustrated and described.
